(19) United States
(12) Patent Application Publication
(10) Pub. No.: US 2026/0283766 A1
(43) Pub. Date: Sep. 24, 2026

(54) DENTAL CLEANING DEVICE

(71) Applicant: Xiamen Aihu Dental Technology Co., Ltd., Xiamen (CN)

(72) Inventor: Zhangsen YOU, Xiamen (CN)

(21) Appl. No.: 19/454,304

(22) Filed: Jan. 20, 2026

(30) Foreign Application Priority Data
Mar. 19, 2025 (CN) 202520481262.8

(51) Int. Cl. A61C 17/024; A61L 2/10

(52) U.S. Cl. CPC A61C 17/024; A61L 2/10; A61L 2202/11

(57) ABSTRACT

A dental cleaning device contains a water tank, a pump module, a sterilization module, a heating module, a water inlet tube, and a handheld water outlet assembly. The water tank has a water storage chamber and an equipment chamber. The heating module is inside the equipment chamber. The sterilization module and the pump module are inside the handheld water outlet assembly. The pump module, used for pumping and ejecting water, is inside the handheld water outlet assembly and is directly connected to the nozzle for enhanced water impacting force. The heating module and the sterilization module can achieve water heating and sterilization effects respectively.

### TECHNICAL FIELD

The present invention relates to a dental cleaning device.

### BACKGROUND OF THE INVENTION

With the improvement in living standards, the emphasis on oral health has also increased. Currently, brushing teeth is the most common method for oral cleaning, but toothbrushes cannot clean the tiny gaps between teeth, leading to the accumulation of plaque and causing dental and gum diseases. As a result, dental cleaning devices have emerged, which use pulsed water jets to clean teeth and interdental spaces, thereby ensuring oral health.

Common dental cleaning devices include handheld, tabletop, or wall-mounted types. Typical tabletop or wall-mounted dental cleaning devices usually feature a large water storage tank and a handheld cleaning handle. The water storage tank is equipped with a water pump, and the cleaning handle is connected to the pump via an approximately 100 cm long water tube. During operation, the pump draws water from the water storage tank and ejects the water through a nozzle of the cleaning handle. However, the long water tube increases the flow path length, reducing the force of the ejected water stream, which can compromise the cleaning effect. Additionally, existing dental cleaning devices often eject cold water, which can irritate teeth in winter, leading to poor user’s experience. Moreover, residual water in these existing dental cleaning devices can breed bacteria which will contaminate the ejected water.

### BRIEF SUMMARY OF THE INVENTION

It is an object of the present invention to provide a dental cleaning device that has good impacting force, and is comfortable to use as well as safe and hygienic.

To attain the above object, the present invention provides the following technical solutions:

A dental cleaning device, comprising a water tank, a pump module, a sterilization module, a heating module, a water inlet tube, and a handheld water outlet assembly;

the water tank is provided with a water storage chamber and an equipment chamber; the heating module is disposed in the equipment chamber; the sterilization module and the pump module are disposed in the handheld water outlet assembly; the handheld water outlet assembly is detachably arranged on one side of the water tank;

the heating module is an instant heating device; a water inlet of the heating module is connected to the water storage chamber, and a water outlet of the heating module is connected to one end of the water inlet tube; another end of the water inlet tube is connected to a water inlet of the sterilization module disposed in the handheld water outlet assembly; a water outlet of the sterilization module is connected to a water inlet of the pump module, and the water outlet of the pump module is connected to a nozzle installed on the handheld water outlet assembly.

Further, the handheld water outlet assembly comprises an oral irrigator housing; the nozzle is detachably mounted on a top side of the oral irrigator housing; the sterilization module and the pump module are installed inside the oral irrigator housing; said another end of the water inlet tube is connected to the water inlet of the sterilization module disposed in the handheld water outlet assembly via a bottom side of the oral irrigator housing.

Further, the dental cleaning device further comprises a suction pump disposed in the equipment chamber and connected between the water outlet of the heating module and said one end of the water inlet tube.

Further, the sterilization module comprises a control mainboard and a transparent flow tube; one end of the transparent flow tube is the water inlet of the sterilization module; another end of the transparent flow tube is the water outlet of the sterilization module; the control mainboard comprises a plurality of sterilization lamps; the plurality of sterilization lamps face towards the transparent flow tube.

Further, the sterilization module also comprises an outer shell and a light-blocking sleeve; the light-blocking sleeve sleeves around a peripheral side of the transparent flow tube, and the outer shell sleeves around a peripheral side of the light-blocking sleeve; the control mainboard is installed on the outer shell; the light-blocking sleeve is provided with a plurality of light-transmitting zones corresponding to the plurality of sterilization lamps to allow the plurality of sterilization lamps to irradiate water inside the transparent flow tube for sterilization.

Further, the transparent flow tube is an elongated glass tube extending vertically; the plurality of sterilization lamps are positioned along an axial direction of the transparent flow tube and being spaced apart from one another; the plurality of light-transmitting zones of the light-blocking sleeve are through holes penetrating through the light-blocking sleeve.

Further, one side of the outer shell of the sterilization module comprises an installation slot in which the control mainboard is mounted; a plurality of lamp holes in communication with an inner cavity of the outer shell are formed in the installation slot; the plurality of sterilization lamps are inserted through the plurality of lamp holes; the light-blocking sleeve is positioned in the inner cavity; the outer shell of the sterilization module comprises an upper opening in communication with the inner cavity, and a bottom of the inner cavity forms an installation stepped portion; the transparent flow tube and the light-blocking sleeve are inserted into the inner cavity from the upper opening; lower ends of the transparent flow tube and the light-blocking sleeve abut against the lower sealing connector and rest on the installation stepped portion; the installation stepped portion comprises a lower hole connected to an external environment; the transparent flow tube is connected to the water inlet tube at the lower hole.

Further, the pump module comprises a housing, a sprayer pump, a motor, a dental cleaning control board, and a battery; the sprayer pump and the motor are installed inside the housing; the motor drives the sprayer pump to operate; a water inlet of the sprayer pump is connected to the water outlet of the sterilization module, and a water outlet of the sprayer pump is connected to the nozzle; the motor and the battery are electrically connected to the dental cleaning control board; the battery is configured to power the motor; the dental cleaning control board controls the motor to operate.

Further, an inner surface of the handheld water outlet assembly comprises a wireless charging module, which is capable of being electrically connected to the battery; said one side of the water tank is recessed to form an accommodating slot that holds the handheld water outlet assembly; an inner side wall of the accommodating slot on said one side of the water tank comprises a wireless charging component; when the handheld water outlet assembly is placed in the accommodating slot, the wireless charging component wirelessly charges the battery.

Further, said one side of the water tank is recessed to form an accommodating slot that holds the handheld water outlet assembly as well as the water inlet tube; the accommodating slot comprises a holding plate that supports the handheld water outlet assembly; the water inlet tube is located below the holding plate;

further, the water tank also comprises a nozzle disinfection chamber; a removable cover is disposed at a top side of the nozzle disinfection chamber; the nozzle disinfection chamber comprises a holed clamping plate with a plurality of clamping holes each being capable to hold the nozzle; a disinfection lamp is installed below the holed clamping plate.

By using the technical solutions given above, the dental cleaning device according to the present invention can be used as either a wall-mounted or a tabletop device either hung on the wall or placed on the tabletop. Compared with prior arts, the handheld water outlet assembly of the present invention is still connected to the water tank via the water inlet tube, but the pump module, which is configured to pump and eject water, is disposed in the handheld water outlet assembly and is directly connected to the nozzle. This effectively enhances the water ejection force from the nozzle, and thus improving the impacting force and hence the cleaning effect. Moreover, the heating module is located between the water storage chamber and the water inlet tube to enable instant and rapid heating of water drawn from the water storage chamber. This ensures that the water ejected out from the nozzle has a temperature comfortable for user to use, avoiding irritation to the mouth, teeth and gums and enhancing user’s experience. Additionally, the sterilization module is also disposed in the handheld water outlet assembly at a position between the water inlet tube and the pump module for sterilizing water before ejection, ensuring clean and hygienic water and preventing oral infections, thereby improving product safety.

### BRIEF DESCRIPTION OF THE DRAWINGS

FIG. 1 is a perspective view of an embodiment of the present invention;

FIG. 2 is an exploded view of an embodiment of the present invention;

FIG. 3 is a top view of an embodiment of the present invention;

FIG. 4 is a cross-sectional view taken along line A-A in FIG. 3;

FIG. 5 is a cross-sectional view taken along line B-B in FIG. 3;

FIG. 6 is an exploded view of a handheld water outlet assembly of an embodiment of the present invention;

FIG. 7 is an exploded view of a sterilization module of an embodiment of the present invention;

FIG. 8 is an exploded view of a pump module of an embodiment of the present invention.

FIG. 9 is a sectional view of the handheld water outlet assembly of an embodiment of the present invention.

### REFERENCE NUMERALS

Water tank 1, water storage chamber 11, equipment chamber 12, accommodating slot 13, holding plate 131, wireless charging component 132, nozzle disinfection chamber 14, removable chamber cover 141, holed clamping plate 142, clamping hole 143, disinfection lamp 144;

Pump module 2, housing 21, sprayer pump 22, motor 23, dental cleaning control board 24, battery 25, water outlet 201 of the pump module, water inlet 202 of the pump module;

Sterilization module 3, outer shell 31, installation slot 311, lamp hole 312, inner cavity 313 of the outer shell, upper opening 314, installation stepped portion 315, lower hole 316 of the outer shell, clamping groove 317, clamping seat 318, control mainboard 32, sterilization lamp 321, light-blocking sleeve 33, light-transmitting zone 331, transparent flow tube 34, lower sealing connector 35, upper sealing connector 36, water outlet 301 of the sterilization module, water inlet 302 of the sterilization module;

Heating module 4, water inlet 41 of the heating module, water outlet 42 of the heating module; water inlet tube 5; handheld water outlet assembly 6, oral irrigator housing 61, cavity 611, base plate 62, wireless charging module 63; nozzle 7, adapter 8, clamping block 81, check valve 9, check piston 91, piston spring 92, suction pump 10.

### DETAILED DESCRIPTION OF THE INVENTION

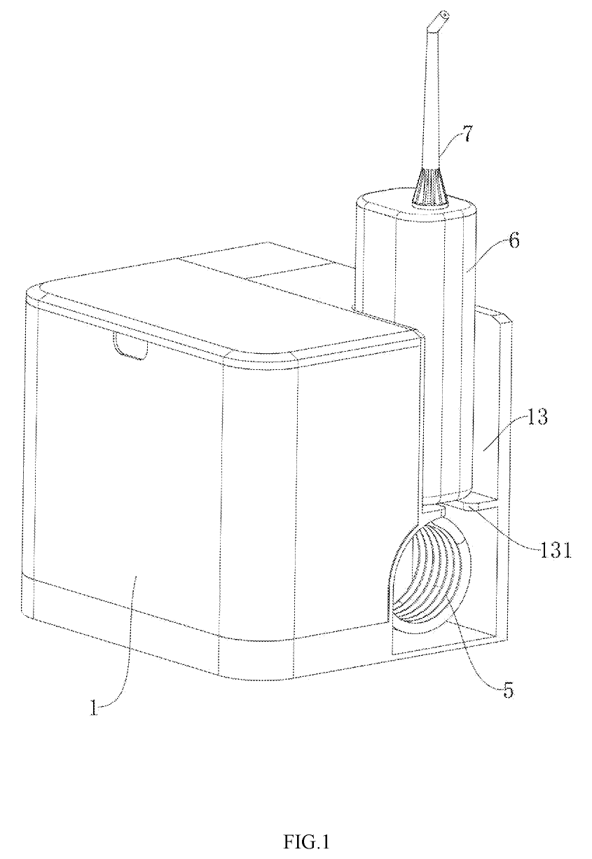

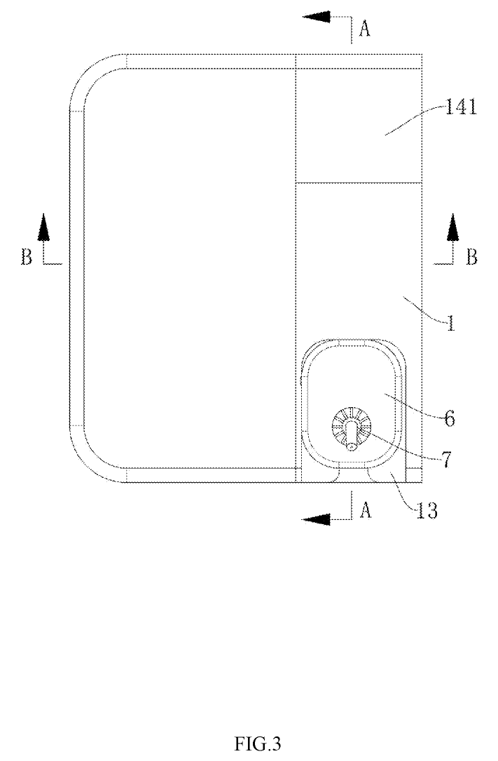

As shown in FIGS. 1 to 5, an embodiment of the dental cleaning device comprises a water tank 1, a pump module 2, a sterilization module 3, a heating module 4, a water inlet tube 5, and a handheld water outlet assembly 6.

The water tank 1 is provided with a water storage chamber 11 and an equipment chamber 12. The heating module 4 is disposed in the equipment chamber 12. The sterilization module 3 and the pump module 2 are disposed in the handheld water outlet assembly 6. The handheld water outlet assembly 6 is detachably arranged on one side of the water tank 1.

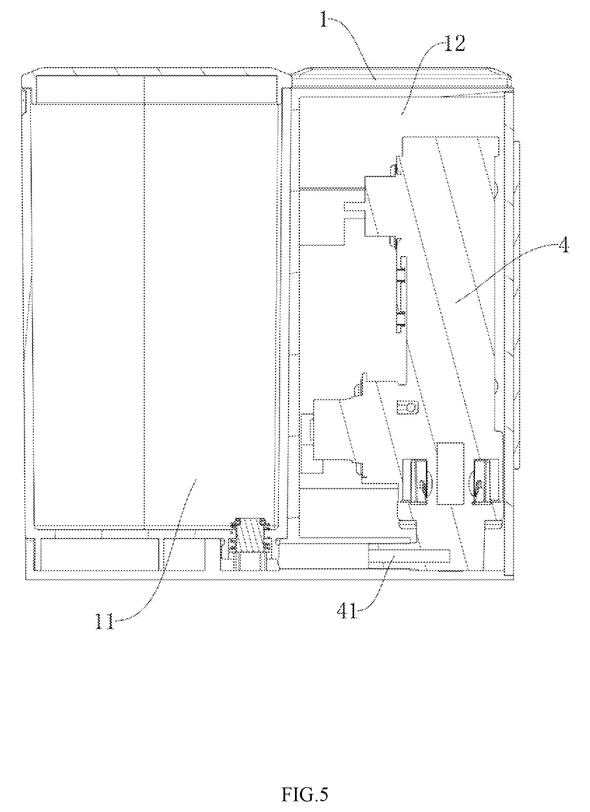

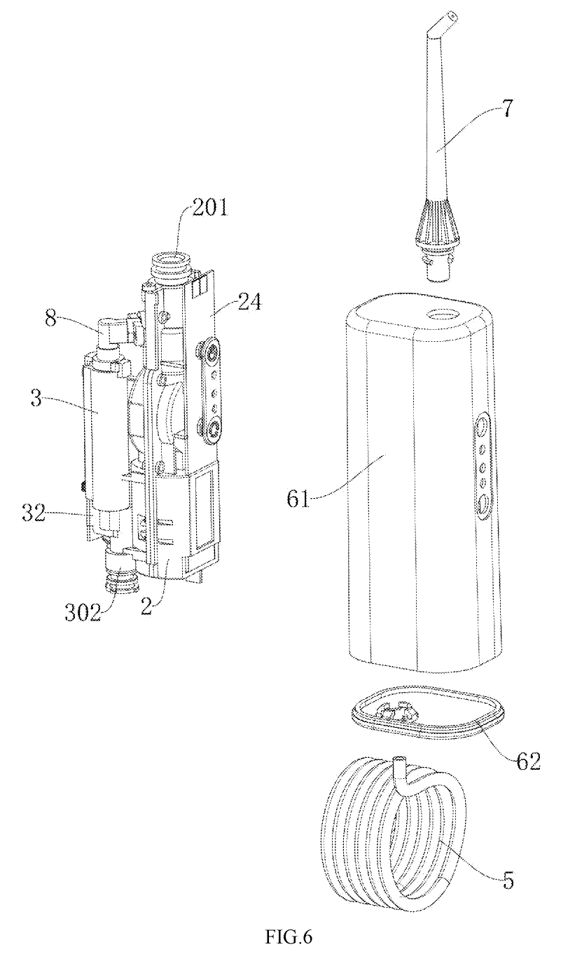

The heating module 4 is an instant heating device. This kind of instant heating device is known in the art and therefore will not be detailed herein. An example of this kind of instant heating device is an instant heating assembly of model number DP006003 manufactured by Fujian Aimi Graphene Technology Co., Ltd. An internal structure of the heating module 4 is not shown in the figures. As shown in FIGS. 5 and 6, a water inlet 41 of the heating module 4 is connected to the water storage chamber 11, and a water outlet 42 of the heating module 4 is connected to one end of the water inlet tube 5. Another end of the water inlet tube 5 is connected to a water inlet 302 of the sterilization module 3 disposed in the handheld water outlet assembly 6. A water outlet 301 of the sterilization module 3 is connected to a water inlet 202 of the pump module 2, and the water outlet 201 of the pump module 2 is connected to a nozzle 7 installed on the handheld water outlet assembly 6.

Thus, the dental cleaning device can be used as either a wall-mounted or tabletop device. In this embodiment, the handheld water outlet assembly 6 is still connected to the water tank 1 via the water inlet tube 5, but the pump module 2, which is configured to pump and eject water, is disposed in the handheld water outlet assembly 6 and is directly connected to the nozzle 7. This effectively enhances the water ejection force from the nozzle 7, and thus improving the impacting force and hence the cleaning effect. Moreover, the heating module 4 is located between the water storage chamber 11 and the water inlet tube 5 to enable instant and rapid heating of water drawn from the water storage chamber 11. This ensures that the water ejected out from the nozzle 7 has a temperature comfortable for user to use, avoiding irritation to the mouth, teeth and gums and enhancing user’s experience. Additionally, the sterilization module 3 is also disposed in the handheld water outlet assembly 6 at a position between the water inlet tube 5 and the pump module 2 for sterilizing water before ejection, ensuring clean and hygienic water and preventing oral infections, thereby improving product safety.

Optionally, the dental cleaning device may further comprises a suction pump 10, which is disposed in the equipment chamber 12 and connected between the water outlet 42 of the heating module 4 and said one end of the water inlet tube 5 to further enhance the water ejection force.

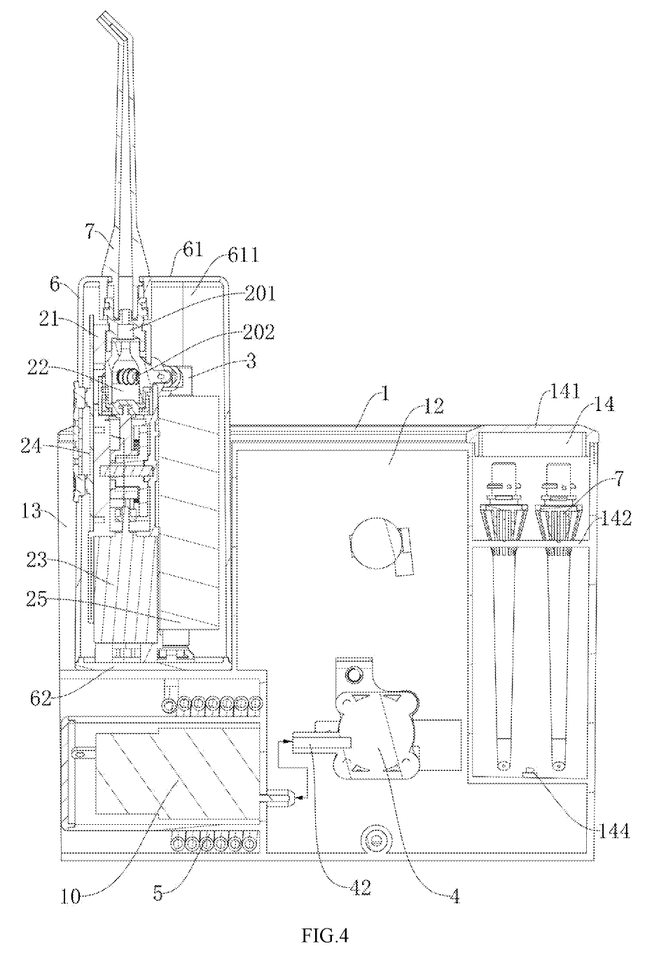

As shown in FIGS. 4 and 6, in this embodiment, the handheld water outlet assembly 6 comprises an oral irrigator housing 61; the nozzle 7 is detachably mounted on a top side of the oral irrigator housing 61; the sterilization module 3 and the pump module 2 are installed inside the oral irrigator housing 61. Said another end of the water inlet tube 5 is connected to the water inlet 302 of the sterilization module 3 inside the handheld water outlet assembly 6 via a bottom side of the oral irrigator housing 61.

The oral irrigator housing 61 may comprise a cavity 611; a base plate 62 is installed at a bottom of the cavity 611. The sterilization module 3 and the pump module 2 are installed in the cavity 611, and the nozzle 7 is mounted on top of the oral irrigator housing 61.

In this embodiment, said one side of the water tank 1 is recessed to form an accommodating slot 13 for holding the handheld water outlet assembly 6. The accommodating slot 13 comprises a holding plate 131 that supports the handheld water outlet assembly 6; the water inlet tube 5 is located below the holding plate 131. Accordingly, it is easy to return and retrieve the handheld water outlet assembly 6 in and out of the water tank 1 to ensure convenient use.

The water inlet tube 5 may be a flexible coiled tube to facilitate mobility of the handheld water outlet assembly 6 to and from a distant location when being taken out for use, and when the handheld water outlet assembly 6 is returned back to the water tank 1, the water inlet tube 5 may quickly resume its coiled shape to facilitate storage.

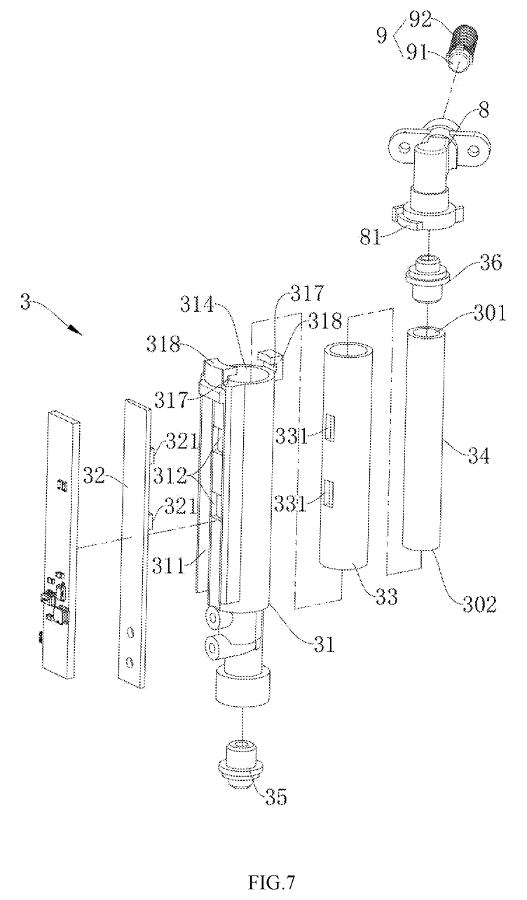

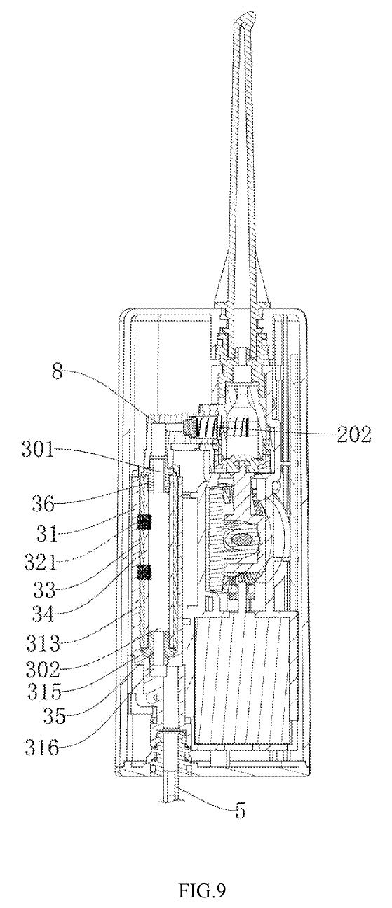

Referring to FIGS. 4, 7 and 9, in this embodiment, the sterilization module 3 comprises an outer shell 31, a control mainboard 32, a light-blocking sleeve 33, and a transparent flow tube 34.

The transparent flow tube 34 may be an elongated tube extending vertically. One end of the transparent flow tube 34 serves as the water inlet 302 of the sterilization module 3 and is connected to said another end of the water inlet tube 5 via a lower sealing connector 35. Said one end of the water inlet tube 5 is connected to the heating module 4. Another end of the transparent flow tube 34 serves as the water outlet 301 of the sterilization module 3 and is connected to one end of an adapter 8 via an upper sealing connector 36. Another end of the adapter 8 is connected to the water inlet 202 of the pump module 2.

The light-blocking sleeve 33 sleeves around a peripheral side of the transparent flow tube 34, and the outer shell 31 sleeves around a peripheral side of the light-blocking sleeve 33. The control mainboard 32 is installed on the outer shell 31 and comprises a plurality of sterilization lamps 321. The light-blocking sleeve 33 is provided with a plurality of light-transmitting zones 331 corresponding to the plurality of sterilization lamps 321 to allow the sterilization lamps 321 to irradiate the water inside the transparent flow tube 34 for sterilization.

Since the transparent flow tube 34 is located between the water inlet tube 5 and the pump module 2, when water is drawn from the storage chamber 11 and flows through the heating module 4 and the water inlet tube 5 into the transparent flow tube 34, the sterilization lamps 321 can quickly and effectively sterilize the water passing through the transparent flow tube. Further, as the light-blocking sleeve 33 around the peripheral side of the transparent flow tube 34, the light-blocking sleeve 33 prevents scattering of sterilization light from the sterilization lamps 321, and thereby enhancing the sterilization effect.

Alternatively, the sterilization module 3 may only comprise the transparent flow tube 34, the control mainboard 32, and the plurality of sterilization lamps 321. Sterilization can still be achieved accordingly.

The transparent flow tube 34 may be an elongated tube, and the plurality of sterilization lamps 321 may be positioned along an axial direction of the transparent flow tube 34 and being spaced apart from one another. This ensures thorough sterilization through a longer time even at a high water flow rate so as to enhance the sterilization effect. In this embodiment, two dual-wavelength UV sterilization lamps 321 are used as examples for enhanced sterilization, capable of killing specific bacteria. Alternatively, single-wavelength sterilization lamps may also be used.

Specifically, one side of the outer shell 31 may comprise an installation slot 311 for mounting the control mainboard 32. The installation slot 311 comprises a plurality of lamp holes 312 in communication with an inner cavity 313 of the outer shell 31; the plurality of sterilization lamps 321 are inserted through the plurality of lamp holes 312. The light-blocking sleeve 33 is positioned in the inner cavity 313. The plurality of light-transmitting zones 331 of the light-blocking sleeve 33 may be through holes penetrating through the light-blocking sleeve 33 to allow the plurality of sterilization lamps 321 to pass through and irradiate the transparent flow tube 34. The transparent flow tube 34, made of high-temperature-resistant glass to withstand heated water, is installed inside the light-blocking sleeve 33.

By sleeving the transparent flow tube 34 and the light-blocking sleeve 33 within the outer shell 31, the control mainboard 32 is separated from the transparent flow tube 34 in which water flows to improve safety and preventing damage to the control mainboard 32 in case of water leakage from the transparent flow tube 34, thereby enhancing product reliability.

The outer shell 31 comprises an upper opening 314 in communication with the inner cavity 313, and a bottom of the inner cavity 313 forms an installation stepped portion 315. The transparent flow tube 34 and the light-blocking sleeve 33 can be inserted into the inner cavity 313 from the upper opening 314; lower ends of the transparent flow tube 34 and the light-blocking sleeve 33 abut against the lower sealing connector 35 and rest on the installation stepped portion 315. The installation stepped portion 315 comprises a lower hole 316 connected to an external environment to facilitate assembly. The transparent flow tube 34 can be connected to the water inlet tube 5 via the lower sealing connector 35 at the lower hole 316.

The upper opening 314 of the outer shell 31 may comprise two clamping seats 318 with two side clamping grooves 317 respectively. The adapter 8 may have an inverted L shape, with two clamping blocks 81 at around said one end of the adapter 8. The adapter 8 can be quickly clamped to the upper opening 314 by rotating the adapter 8 during installation to the upper opening 314 such that the clamping blocks 81 slide into the clamping grooves 317. The upper sealing connector 36 is placed between the upper opening 314 and said one end of the adapter 8 to achieve a sealing effect between the upper opening 314 and said one end of the adapter 8.

In this embodiment, the sterilization module 3 and the pump module 2 are arranged side by side. Said another end of the adapter defined as a right opening of the adapter 8 is connected to the water inlet 202 of the pump module 2.

The right opening of the adapter 8 may also be connected to a check valve 9 to ensure one-way water flow from the sterilization module 3 to the pump module 2, preventing backflow of water from the nozzle 7. The check valve 9 comprises a check piston 91 and a piston spring 92.

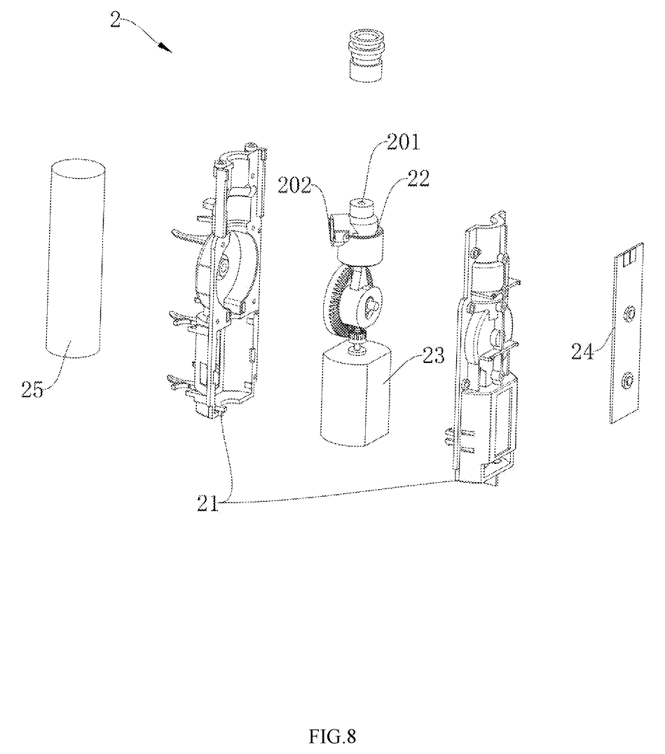

As shown in FIGS. 4 and 8, the pump module 2 comprises a housing 21, a sprayer pump 22, a motor 23, a dental cleaning control board 24, and a battery 25. The sprayer pump 22 and the motor 23 are installed inside the housing 21; the motor 23 drives the sprayer pump 22 to operate. A water inlet (i.e. which is the water inlet 202 of the pump module 2) of the sprayer pump 22 is connected to the right opening of the adapter 8, and a water outlet (i.e. the water outlet 201 of the pump module 2) of the sprayer pump 22 is connected to the nozzle 7. The battery 25 and the dental cleaning control board 24 are installed on two opposite sides of the housing 21 respectively. The motor 23 and the battery 25 are electrically connected to the dental cleaning control board 24. The battery 25 is configured to power the motor 23; the dental cleaning control board 24 controls the motor 23 to operate.

The control mainboard 32 may also be electrically connected to the dental cleaning control board 24, allowing the dental cleaning control board 24 to control the plurality of sterilization lamps 321.

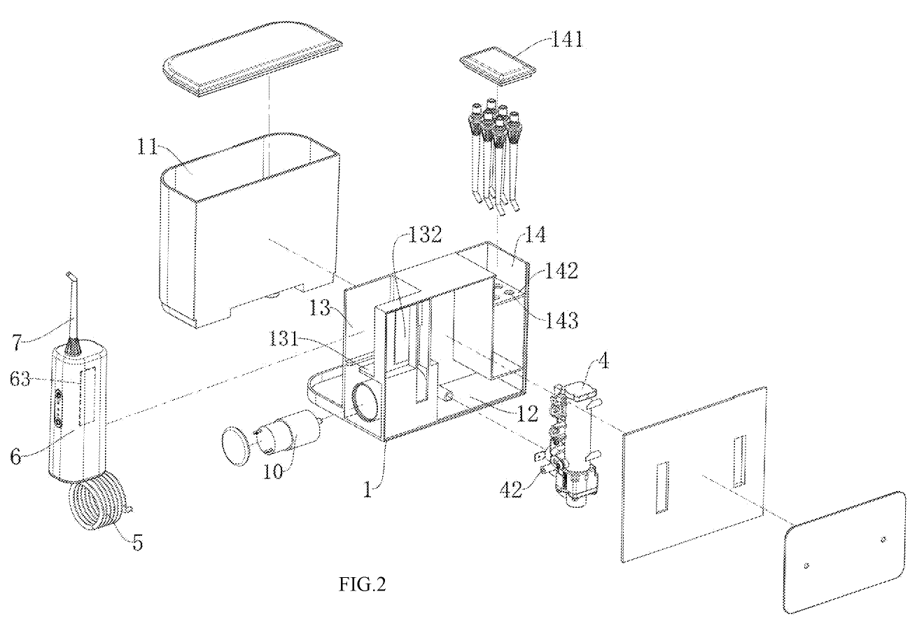

As shown in FIG. 2, an inner surface of the handheld water outlet assembly 6 may comprise a wireless charging module 63, which is electrically connected to the battery 25. An inner side wall of the accommodating slot 13 on said one side of the water tank 1 may comprise a wireless charging component 132. When the handheld water outlet assembly 6 is placed in the accommodating slot 13, the wireless charging component 132 wirelessly charges the battery 25.

The wireless charging module 63 and the wireless charging component 132 may be charging coils.

As shown in FIG. 2, the water tank 1 also comprises a nozzle disinfection chamber 14; a removable cover 141 is disposed at a top side of the nozzle disinfection chamber 14. The nozzle disinfection chamber 14 comprises a holed clamping plate 142 with a plurality of clamping holes 143 for holding nozzles. A disinfection lamp 144 is installed below the holed clamping plate 142, allowing for storage and sterilization of the nozzle 7 after use. The disinfection lamp 144 may also be a dual-wavelength UV sterilization lamp.

In this embodiment, the water tank 1 may be made of PP, ABS, PET, or similar materials with lubricating additives to achieve a lotus effect, preventing water droplets from adhering to the inner walls of the water tank 1 and inhibiting bacterial growth, thereby enhancing hygiene and safety.

What is claimed is:

1. A dental cleaning device, comprising a water tank, a pump module, a sterilization module, a heating module, a water inlet tube, and a handheld water outlet assembly;
the water tank is provided with a water storage chamber and an equipment chamber; the heating module is disposed in the equipment chamber; the sterilization module and the pump module are disposed in the handheld water outlet assembly; the handheld water outlet assembly is detachably arranged on one side of the water tank;
the heating module is an instant heating device; a water inlet of the heating module is connected to the water storage chamber, and a water outlet of the heating module is connected to one end of the water inlet tube; another end of the water inlet tube is connected to a water inlet of the sterilization module disposed in the handheld water outlet assembly; a water outlet of the sterilization module is connected to a water inlet of the pump module, and the water outlet of the pump module is connected to a nozzle installed on the handheld water outlet assembly.

2. The dental cleaning device of claim 1, wherein the handheld water outlet assembly comprises an oral irrigator housing; the nozzle is detachably mounted on a top side of the oral irrigator housing; the sterilization module and the pump module are installed inside the oral irrigator housing; said another end of the water inlet tube is connected to the water inlet of the sterilization module disposed in the handheld water outlet assembly via a bottom side of the oral irrigator housing.

3. The dental cleaning device of claim 1, further comprising a suction pump disposed in the equipment chamber and connected between the water outlet of the heating module and said one end of the water inlet tube.

4. The dental cleaning device of claim 1, wherein the sterilization module comprises a control mainboard and a transparent flow tube; one end of the transparent flow tube is the water inlet of the sterilization module; another end of the transparent flow tube is the water outlet of the sterilization module; the control mainboard comprises a plurality of sterilization lamps; the plurality of sterilization lamps face towards the transparent flow tube.

5. The dental cleaning device of claim 4, wherein the sterilization module also comprises an outer shell and a light-blocking sleeve; the light-blocking sleeve sleeves around a peripheral side of the transparent flow tube, and the outer shell sleeves around a peripheral side of the light-blocking sleeve; the control mainboard is installed on the outer shell; the light-blocking sleeve is provided with a plurality of light-transmitting zones corresponding to the plurality of sterilization lamps to allow the plurality of sterilization lamps to irradiate water inside the transparent flow tube for sterilization.

6. The dental cleaning device of claim 4, wherein the transparent flow tube is an elongated glass tube extending vertically; the plurality of sterilization lamps are positioned along an axial direction of the transparent flow tube and being spaced apart from one another; the plurality of light-transmitting zones of the light-blocking sleeve are through holes penetrating through the light-blocking sleeve.

7. The dental cleaning device of claim 5, wherein one side of the outer shell of the sterilization module comprises an installation slot in which the control mainboard is mounted; a plurality of lamp holes in communication with an inner cavity of the outer shell are formed in the installation slot; the plurality of sterilization lamps are inserted through the plurality of lamp holes; the light-blocking sleeve is positioned in the inner cavity; the outer shell of the sterilization module comprises an upper opening in communication with the inner cavity, and a bottom of the inner cavity forms an installation stepped portion; the transparent flow tube and the light-blocking sleeve are inserted into the inner cavity from the upper opening; lower ends of the transparent flow tube and the light-blocking sleeve abut against the lower sealing connector and rest on the installation stepped portion; the installation stepped portion comprises a lower hole connected to an external environment; the transparent flow tube is connected to the water inlet tube at the lower hole.

8. The dental cleaning device of claim 1, wherein the pump module comprises a housing, a sprayer pump, a motor, a dental cleaning control board, and a battery; the sprayer pump and the motor are installed inside the housing; the motor drives the sprayer pump to operate; a water inlet of the sprayer pump is connected to the water outlet of the sterilization module, and a water outlet of the sprayer pump is connected to the nozzle; the motor and the battery are electrically connected to the dental cleaning control board; the battery is configured to power the motor; the dental cleaning control board controls the motor to operate.

9. The dental cleaning device of claim 8, wherein an inner surface of the handheld water outlet assembly comprises a wireless charging module, which is capable of being electrically connected to the battery; said one side of the water tank is recessed to form an accommodating slot that holds the handheld water outlet assembly; an inner side wall of the accommodating slot on said one side of the water tank comprises a wireless charging component; when the handheld water outlet assembly is placed in the accommodating slot, the wireless charging component wirelessly charges the battery.

10. The dental cleaning device of claim 1, wherein said one side of the water tank is recessed to form an accommodating slot that holds the handheld water outlet assembly as well as the water inlet tube; the accommodating slot comprises a holding plate that supports the handheld water outlet assembly; the water inlet tube is located below the holding plate; the water tank also comprises a nozzle disinfection chamber; a removable cover is disposed at a top side of the nozzle disinfection chamber; the nozzle disinfection chamber comprises a holed clamping plate with a plurality of clamping holes each being capable to hold the nozzle; a disinfection lamp is installed below the holed clamping plate.
